(19) United States
(12) Patent Application Publication
(10) Pub. No.: US 2013/0012474 A1
(43) Pub. Date: Jan. 10, 2013

(54) Cellulose Gel Formulations

(75) Inventors: Albert Mihranyan, Uppsala (SE); Maria Strömme, Uppsala (SE)

(73) Assignee: FMC CORPORATION, Philadelphia, PA (US)

(21) Appl. No.: 13/618,235

(22) Filed: Sep. 14, 2012

Related U.S. Application Data
(62) Division of application No. 12/096,047, filed on Oct. 9, 2008.
(60) Provisional application No. 60/742,749, filed on Dec. 6, 2005.

(51) Int. Cl. C08B 15/00; A23L 1/0534; A61K 31/717; C09D 101/00; A61K 47/38; A61K 8/73

(52) U.S. Cl. 514/57; 536/30; 514/781; 106/163.01; 426/506

(57) ABSTRACT

The invention relates to dispersible cellulose powder compositions comprising non-seed cellulose powder derived from algae, fungi or tunicates, which compositions are useful in a variety of products such as food products, pharmaceuticals, cosmetics, paints, biocompatible materials for artificial tissue engineering and implantable biomaterials and relates to methods for preparing non-seed cellulose powder compositions.

### FIELD OF INVENTION

The invention relates to dispersible cellulose powder compositions comprising non-seed cellulose powder derived from algae, fungi or tunicates, which compositions are useful in a variety of products, for example, food products, pharmaceuticals, cosmetics, paints, biocompatible materials for artificial tissue engineering and implantable biomaterials. The invention also relates to methods for preparing non-seed cellulose powder compositions.

### BACKGROUND OF THE INVENTION

Microcrystalline cellulose (MCC) is an additive commonly used for various industrial applications including food, drugs and cosmetic products. It is defined as a purified, partly depolymerized cellulose prepared by treating α-cellulose, obtained as a pulp from fibrous plant material, with mineral acid. The term α-cellulose refers to that portion of industrial cellulose pulps which is insoluble in cold sodium hydroxide of mercerisizing strength (17.5 or 18%). β-cellulose is soluble in such a solution but is precipitated upon acidification, while γ-cellulose remains in solution upon acidification.

The MCC particles are primarily aggregates and are composed of millions of crystallites. The crystallites of MCC possess a highly useful property of forming stable homogeneous dispersions which can significantly enhance the body, texture, and stability of other dispersive systems such as suspensions, lotions, creams, ointments, pastes and dairy type comestibles (e.g. ice cream, yogurt, etc). Unlike the water soluble polymers used as thickening agents, the crystallites of MCC are water insoluble, rendering its dispersions with the desirable properties of heat and freeze-thaw stability. Other desirable properties of its dispersions are: long shelf-life stability, stability at a pH range between 4-11, thixotropic, odorless, and tasteless.

Even with these desirable properties, conventional dispersible cellulose grades have been unsatisfactory when relatively large amounts of cellulose are necessary to achieve desired texture and functionality of the final product. These adverse effects are predominantly associated with drying sensation, chalkiness and other undesired organoleptic effects. In addition, the commercially available dispersible cellulose grades exhibit limited electrolyte capacity and readily coagulate in presence of excessive amounts of ionic matter, which is a significant shortcoming as most of the alimentary, pharmaceutical or cosmetic products have complex formulae and contain large proportions of charged species, including both active ingredients and various additives (i.e. preservatives, etc). Accordingly, there remains a need for improved dispersible cellulose grades.

### SUMMARY OF INVENTION

Embodiments of the present invention are directed to dispersible cellulose powder compositions, comprising a non-seed cellulose powder, wherein the non-seed cellulose powder is derived from algae, fungi or tunicates.

Embodiments of the present invention are also directed to gels, suspensions, food products, pharmaceuticals, cosmetics, paints, biocompatible materials for artificial tissue engineering and implantable biomaterials comprising a dispersible cellulose powder composition.

Embodiments of the present invention are further directed to methods for preparing non-seed cellulose powder compositions comprising: purifying a non-seed cellulose mass and co-spray-drying the ground non-seed cellulose mass with a stabilizing agent to form a non-seed cellulose powder composition.

Embodiments of the present invention are further directed to methods for preparing non-seed cellulose powder compositions comprising: purifying a non-seed cellulose mass; grinding a purified non-seed cellulose mass; spray-drying the ground non-seed cellulose; and dispersing the non-seed cellulose composition in a stabilizing agent solution to form a non-seed cellulose powder composition.

### BRIEF DESCRIPTION OF FIGURES

FIG. 1 is a scanning electron microscopy picture of the Cladophora cellulose particle. The displayed surface area value is obtained from N₂ BET gas adsorption analysis.

FIGS. 2 A-B are graphs depicting: A) the elastic modulus G′, obtained at the frequency of 1 Hz, for cellulose samples as a function of their concentration and B) the viscous modulus G″, obtained at the frequency of 1 Hz, for cellulose samples as a function of their concentration.

FIGS. 3A-E are graphs depicting the frequency dependence of the elastic modulus G′ (closed symbols) and the viscous modulus G″ (open symbols) of cellulose powder samples at different concentrations: A) Avicel RC-591 sample, B) Cladophora cellulose sample in water (without addition of CMC), C) Cladophora cellulose in 0.025% (w/v) CMC solution, D) Cladophora cellulose in 0.050% (w/v) CMC solution and E) Cladophora cellulose in 0.100% (w/v) CMC solution.

FIG. 4 is a graph depicting the phase angle δ, obtained at frequency of 1 Hz, for cellulose samples as a function of their concentration.

FIGS. 5 A-E are graphs depicting Cox-Merz complex dynamic viscosity as a function of applied frequency: A) Cladophora cellulose sample in water (without addition of CMC), B) Cladophora cellulose in 0.025% (w/v) CMC solution, C) Cladophora cellulose in 0.05% (w/v) CMC solution, D) Cladophora cellulose in 0.10% (w/v) CMC solution and E) RC-591 sample in water. The error bars denote standard deviations over three measurements.

FIG. 6 is a graph depicting the frequency dependence of the elastic modulus G′ (closed symbols) and the viscous modulus G″ (open symbols) of Vivapur MCG powder, Vivapur wet cake/CMC and Cladophora/CMC samples.

FIG. 7 is a graph depicting Relative Transparency of activated Cladophora cellulose dispersion (5.7±0.3 mg/10 ml) as a function of sonication time. I=light transmission through suspension (%), I₀=light transmission through water (%).

### DETAILED DESCRIPTION OF THE PREFERRED EMBODIMENTS

Traditionally, dispersible cellulose materials are derived from higher plant sources, herein referred to as seed organisms (e.g. wood, plants, etc). However, alternative sources for α-cellulose production are also known, herein referred to as non-seed organisms (e.g. algae, bacteria, fungi). In prior art, cellulose powders of bacterial origin produced from aerobic fermentation of Acetobacter under special agitation conditions are disclosed in U.S. Pat. Nos. 5,079,162, 5,144,021 and 5,366,750 as suitable dispersive cellulose material for food products. However, there is no reference to algal or other non-seed organism origin as a suitable dispersive cellulose material. The rheological properties of tunicate cellulose are described in M. Bercea, P. Navard. 2000. “Shear dynamics of aqueous suspensions of cellulose whiskers”, Macromolecules, 33, 6011-6016. However, no reference to possible applications is indicated.

The inventors have determined that improved cellulose powder compositions may be produced from non-seed cellulose powder. Accordingly, embodiments of the present invention are directed to cellulose powder compositions comprising a non-seed cellulose powder, wherein the non-seed cellulose powder is derived from algae, fungi and/or tunicates. One skilled in the art will appreciate the various algae, fungi or tunicates in which the non-seed cellulose powder may be derived, any of which may be employed herein. For example, the cellulose of algal origin may be cellulose obtained from filamentous and/or spherical marine algae, such as from: Green algae (Chlorophyta): in particular Cladophorales order, e.g. Cladophora, Chaetomorpha, Rhizoclonium, or Microdyction, and Siphonocladales order, e.g. Valonia, Dictyosphaeria, Siphonocladus, or Boergesenia. Also, Green algae (Chlorophyta), such as from Ulvales order, e.g. Ulva, Enteromorpha, Charales order, e.g. Chara, Nitella, Zygnematales order, e.g. Spirogyra, and Chlorococcales order, e.g. Oocystis; Blue green algae (Cyanophyta), such as Anabaena and Nostoc punctiformae; Gold algae (Chrysophyta), such as Vaucheriales order, e.g. Vaucheria, and Tribonematales order, e.g. Tribonema; Dinoflagellates (Pyrrophyta), such as Cryptecodinium cohnii, Gonyaulax, polyedra, Scrippsiella hexapraecingula, Dinobryon and Peridinium; Brown algae (Phaeophyta), such as Lessonia negriscens, Macrocystis pyrifera, Ascophyllum nodosum and Fucus serratus; and Red algae (Rhodophyta), such as Erythrocladia subintegra. Cellulose from fungi may be obtained from fungi selected from Achlya bisexualis; Colletotrichum lindemuthianum; Dictyostelium, such as discoideum; Microdochium nivale; Ophiostoma ulmi; Phytophtora, such as parasitica var. nicotianae and cactorum; Phytium, such as aphanidermatum, butleri and ultimatum; and Saprolegnia, such as parasitica and monoica.

Chemically identical, α-cellulose obtained from seed and non-seed organisms may significantly differ with respect to its supra-molecular order. The width of cellulose crystallites of seed organism origin is typically about 4-5 nm, whereas that of non-seed organism origin is about 20 nm. These differences could be traced to the cellulose synthase complexes that determine the size and shape of cellulose crystallites. In all seed organisms, the cellulose synthases appear as solitary rosettes of six hexagonally arranged subunits, producing thin crystallites. In contrast, synthases of certain non-seed organisms are arranged in large rectangular complexes rather than rosettes and are capable of producing extremely thick crystallites. It is commonly recognized that in algae and bacteria cellulose, Ia is the dominant allomorph of native cellulose, whereas cellulose Iβ is dominant in higher plants. In many algae, where cellulose I is present in the native walls, its X-ray diagram is strikingly sharp, usually revealing a remarkably high degree of structural organization, e.g. Cladophora, Valonia, Microdictyon, etc.

It is believed that the large surface area of cellulose obtained from non-seed organism origin is an important parameter. It is not possible to manufacture seed origin cellulose with similar characteristics to non-seed cellulose by simply spray-drying a well-ground seed organism cellulose suspension with high surface area. The seed cellulose will agglomerate upon drying and give essentially non-porous particles. Even if the cellulose porosity is preserved during drying by physico-chemical methods, the structure is unstable and readily collapses in moist environment. A drastic decrease is found when such cellulose is exposed to humid environment (See K. Matsumoto, Y. Nakai, E. Yonemochi, T. Oguchi, K. Yamamoto. 1998. “Effect of pore size on the gaseous adsorption of ethenzamide on porous crystalline cellulose and the physicochemical stability of ethenzamide after storage.” Chem Pharm Bull, 46 (2), 314-318). As an example, the specific surface area of Cladophora cellulose is close to the surface area of industrial adsorbents. The latter have surface areas of the order of about 100-1000 m²/g. Accordingly, in one embodiment, the surface area of the non-seed cellulose powder is greater than or equal to 5 m²/g. In another embodiment, the surface area of the non-seed cellulose powder is greater than or equal to 8 m²/g.

Traditionally, dispersible cellulose powder is obtained from cell walls of seed organism sources via acidic hydrolysis. The residue is collected as a filter cake and is thoroughly washed to remove soluble impurities. The resultant product is then attrited by means of high shear rubbing in presence of an aqueous medium. During the disintegration, new surfaces are formed as the crystallites are separated, and, unless the individual crystallites are maintained in a separated condition, they will re-bond. It should be emphasized that the particle size distribution is of crucial importance: The attrition should be sufficient to produce a mass wherein at least 1% by weight of solids and preferably at least 30% of the particles do not exceed 1 μm in length as determined by electron microscopy.

For practical purposes, it is important to have a powdered product. However, the crystallites will re-agglomerate upon drying producing an essentially non-porous, low surface area product. Accordingly, in order to prevent re-agglomeration of attrited crystallites, various stabilizing agents may be added to the non-seed cellulose powder composition and one skilled in the art will appreciate the amount of stabilizing agent to be added to the non-seed cellulose powder composition. In one embodiment, a hydrocolloid, such as, carboxymethylcellulose (CMC), guam gum, locust beam gum, gum arabic, sodium alginate, propylene glycol alginate, carrageenan, gum karaya, xanthan or combinations thereof may added to the non-seed cellulose powder composition as a stabilizing agent. In certain embodiments, stabilizing agents may also be referred to as chaotropic agents. The stabilizing action of dispersible cellulose is rendered via steric stabilization. For example, negatively charged stabilizing agent molecules, sitting on the MCC crystallites, are believed to assist the dispersion due to the weak repulsive particle-particle interactions. Hence, the role of the stabilizing agent in the formulation is to both aid the dispersion and also to serve as a protective colloid. Accordingly, one skilled in the art will appreciate that the choice of the stabilizing agent(s) used in the in the non-seed cellulose powder composition depends on a number of factors including, but not limited to, solubility, drying characteristics, application characteristics, and cost.

Functional ingredients may also be added to the non-seed cellulose powder composition to impart, for example, desirable taste, appearance, textural and/or other properties. One skilled in the art will appreciate the various functional ingredients that may be added to the non-seed cellulose powder composition, any of which may be employed herein. Examples include, but are not limited to, flavoring materials, taste modifiers, colorants, humectants, pharmaceutical ingredients, pharmaceutical excipients, one or more biocompatible materials for artificial tissue engineering or combinations of functional ingredients. Moreover, one skilled in the art will appreciate the amount of the functional ingredient(s) to add to the non-seed cellulose powder composition to provide the composition with the desired property.

Embodiments of the present invention are also directed to methods for preparing a non-seed cellulose powder composition. In one embodiment, the methods comprise purifying a non-seed cellulose mass and co-spray-drying the ground non-seed cellulose mass with a stabilizing agent to form a non-seed cellulose powder composition. One skilled in the art will appreciate the various methods for purifying a non-seed cellulose mass, any of which methods may be employed herein. In one embodiment, the step of purifying a non-seed cellulose mass comprises bleaching a non-seed cellulose mass with sodium chlorite and alkali extraction of a-cellulose. Such purifying steps may be performed in a single step or repeated as desired.

Embodiments of the present invention are also directed to methods for preparing a non-seed cellulose composition. The methods comprise: purifying a non-seed cellulose mass; grinding a purified non-seed cellulose mass; spray-drying the ground non-seed cellulose; and dispersing the non-seed cellulose composition in a stabilizing agent solution to prepare the non-seed cellulose composition.

Additional steps may be employed in the methods for preparing a non-seed cellulose powder composition to product different grades of non-seed cellulose. In one embodiment, the method of preparing the non-seed cellulose powder composition may further comprise a step of mechanical comminution (wet or dry) of the non-seed cellulose mass prior to the co-spray drying in which the co-spray drying produces powdered grade of cellulose. In another embodiment, the method of preparing the non-seed cellulose powder composition may further comprise a step of acid hydrolysis of the non-seed cellulose mass prior to co-spray drying, wherein the co-spray drying produces microcrystalline grade of cellulose. In yet another embodiment, the method of preparing the non-seed cellulose powder composition may further comprise a step of activating the non-seed cellulose composition in an aqueous medium using a high-shear homogenizer.

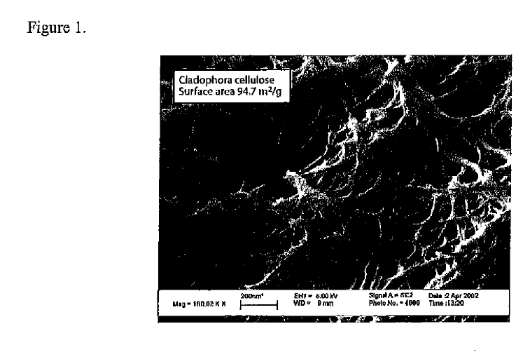

In FIG. 1, a typical web-like structure composed of numerous intertwined cellulose “threads” of around 20-30 nm in width is visible. These “threads” are dispersed in an aqueous medium (containing 0, 0.025, 0.05 and 0.10% (w/v) CMC) using a high intensity ultrasonic processor which would allow quick (within minutes) dispersion in small liquid volumes. However, any other more conventional dispersing technique may also be utilized, as discussed in detail below. The Cladophora cellulose is produced and the gelling properties are compared with a commercial MCC/CMC product, Avicel RC-591 (FMC Corp., US) or Vivapur MCG (JRS Pharma, Germany).

Embodiments of the present invention are also directed to gels and suspensions comprising a non-seed cellulose powder composition. Herein, gel is defined as a soft, solid or solid-like material which consists of at least two components, one of which is a liquid present in abundance (see K. Almdal, J. Dyre, S. Hvidt, and O. Kramer. 1993. “Towards a phenomenological definition of the term ‘gel’”. Polymer Gels and Networks, 1, 5-17).

The gelling properties are described in terms of two dynamic mechanical properties: an elastic modulus G′, which reflects the reversibly stored energy of the system, and a viscous modulus G″, which reflects the irreversible energy loss. When plotted against frequency, a pronounced plateau is exhibited by the G′ modulus for true gel structures. Also, G″ is considerably smaller than G′ in the plateau region. The ratio between G″ and G′ is another measure of viscoelastic properties of gels and is defined as follows:

$$tan δ = \frac{{G}^{″}}{{G}^{′}} \qquad ( 1 )$$

where δ is the phase angle (for elastic structures a δ→0°, whereas for plastic structures δ→90°. According to the Cox-Merz empirical rule (Cox, W. P. and Merz, E. H. 1958. Correlation of dynamic and steady flow viscosities. Journal of Polymer Science, 28, 619-622.), which correlates the steady flow viscosity with the dynamic viscosity, for gel structures the value the complex dynamic viscosity is a monotonically decreasing function of applied frequency. The complex dynamic viscosity is calculated as follows:

$${η}^{*} = {[ {( {η}^{′} )}^{2} + {( \frac{{G}^{′}}{w} )}^{2} ]}^{\frac{1}{2}} \qquad ( 1 )$$

where η* is the complex dynamic viscosity, η′ is the dynamic viscosity, G′ is the dynamic rigidity, and w is the circular frequency.

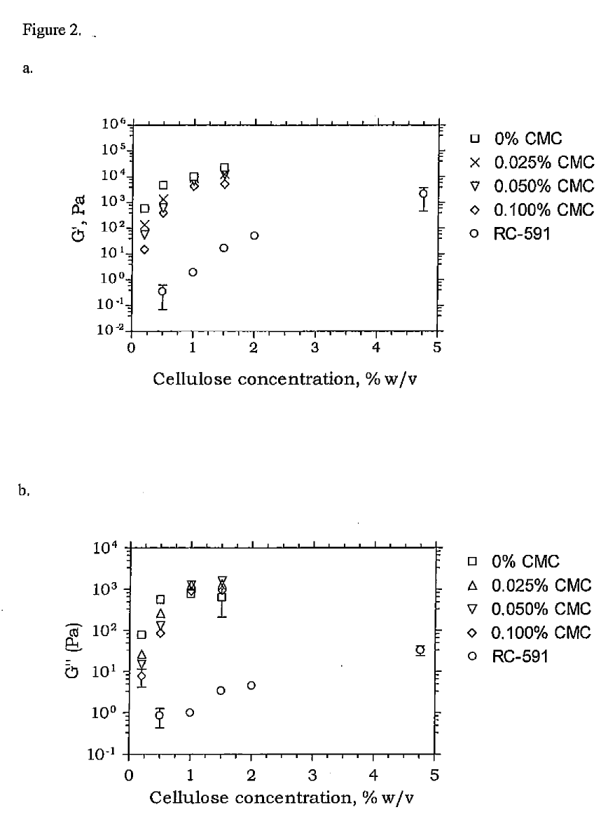

The gel strength of the preparations, described by the elastic modulus G′ at a frequency of 1 Hz, is shown in FIG. 2 as a function of the cellulose concentration. The elastic modulus G′ increased with increasing solid content. Approximately 10 times larger concentration of Avicel RC-591 is needed in order to achieve comparable gel strength as that of the Cladophora samples. For Cladophora solid contents below 0.5% (w/v), the elastic modulus G′ at 1 Hz is in the interval between 10 and 10⁴ Pa for CMC solutions below 0.10% (w/v). For Cladophora solid contents in the interval between 0.5 and 2% (w/v), the elastic modulus G′ at x Hz is in the interval between 10² and 10⁵ Pa for CMC solutions below 0.10% (w/v).

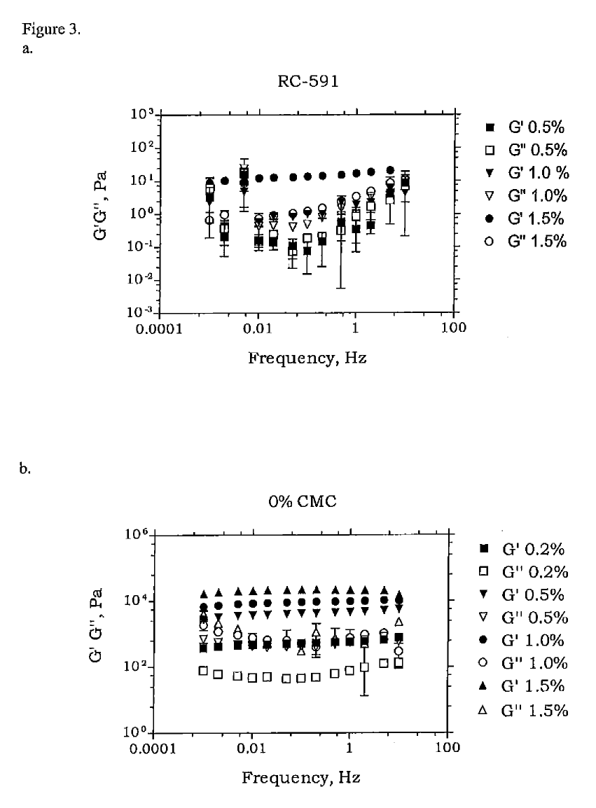

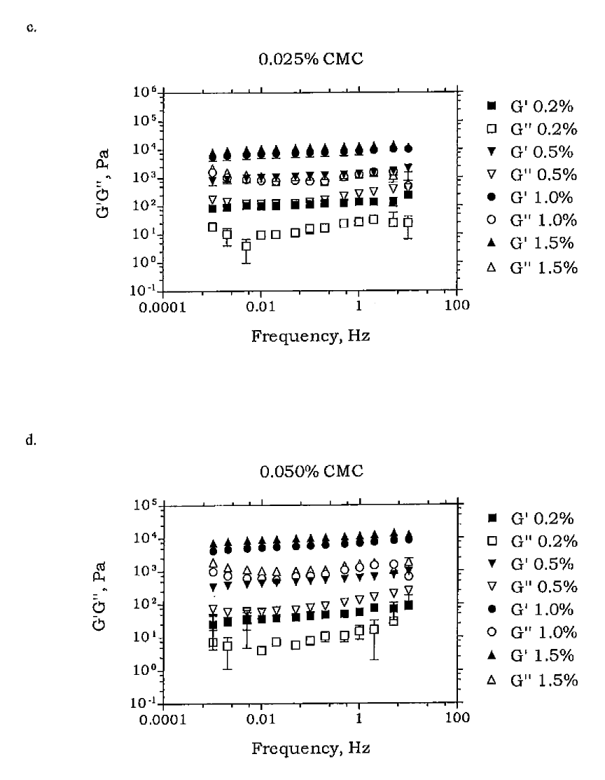

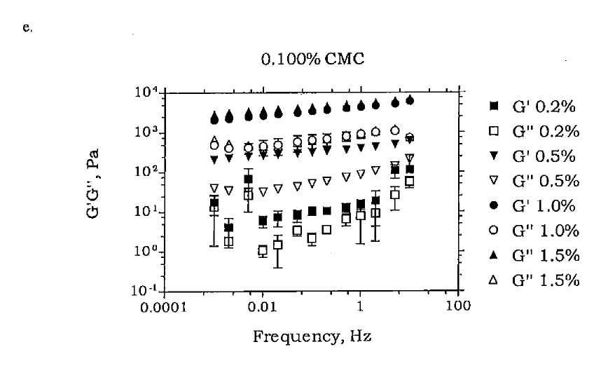

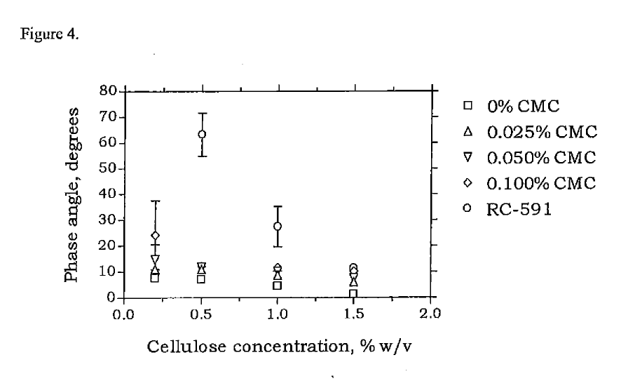

In FIG. 3, the data of the oscillation sweep measurements are summarized. From FIG. 3a, it can be concluded that Avicel RC-591 does not form gel structures at concentrations less than 1.5% w/v solid. This conclusion is based on the frequency dependent pattern of the G′ component. It is also supported by the high values of the phase angle 6 in FIG. 4 for Avicel RC-591 concentrations of 0.5 and 1.0% w/v. On the other hand, for Avicel RC-591 of 1.5% w/v concentration a frequency independent G′ modulus, FIG. 3a, as well as low values of the phase angle δ˜10°, FIG. 4, are observed; however, generally low values of G′ and G″ suggest a weak gel structure. Similarly, 0.2% w/v solids content Cladophora sample prepared using 0.100% w/v CMC solution exhibit rheological properties typical for a viscous system rather than those for an elastic gel. This is evident from the frequency dependent character of the G′ modulus, FIG. 3e, and relatively high value of the phase angle δ, FIG. 4. For the rest of the Cladophora samples, at all measured concentrations, a frequency independent G′ component is observed, FIG. 3b-e. The phase angle δ values of about 10° and less are also registered, FIG. 4, recognized as characteristic for elastic gel structures. Relatively high values for the G′ and G″ moduli of the Cladophora samples suggested firm gel structures characterized by strong interactions over long distances.

The rheological analysis show weaker gel structures as the concentration of CMC is increased, especially for 0.100% w/v CMC solutions, FIG. 3b-e. It should be noted that the influence of CMC concentration on gelling properties of the Cladophora cellulose powder is more pronounced at lower solid contents, e.g. 0.2 and 0.5% w/v, whereas at higher solid concentrations the differences are almost negligible, FIG. 2. Even though CMC has a negative effect on the gel strength of Cladophora cellulose, its addition in small amounts is found useful to aid the dispersion since more homogeneous products are obtained as observed visually.

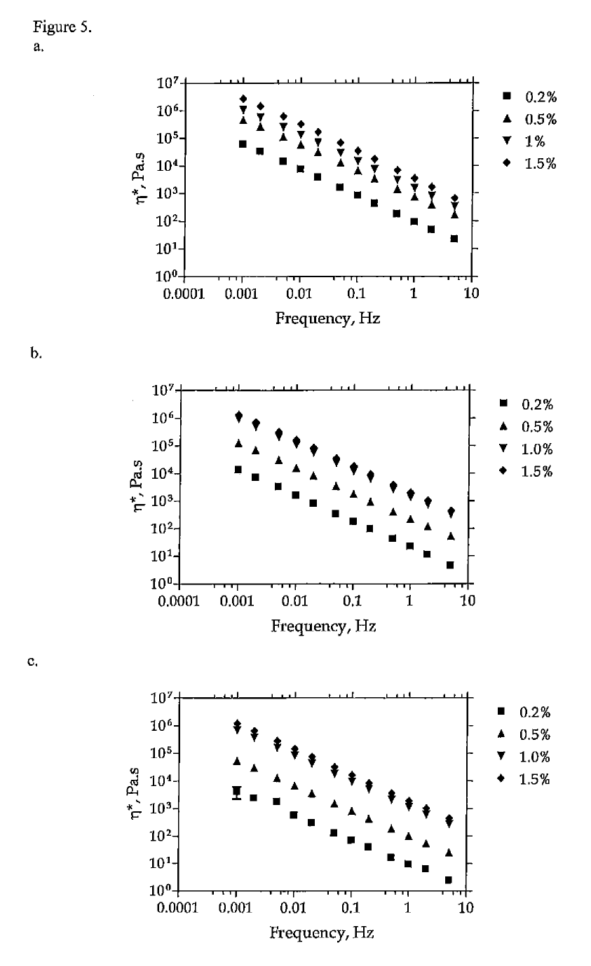

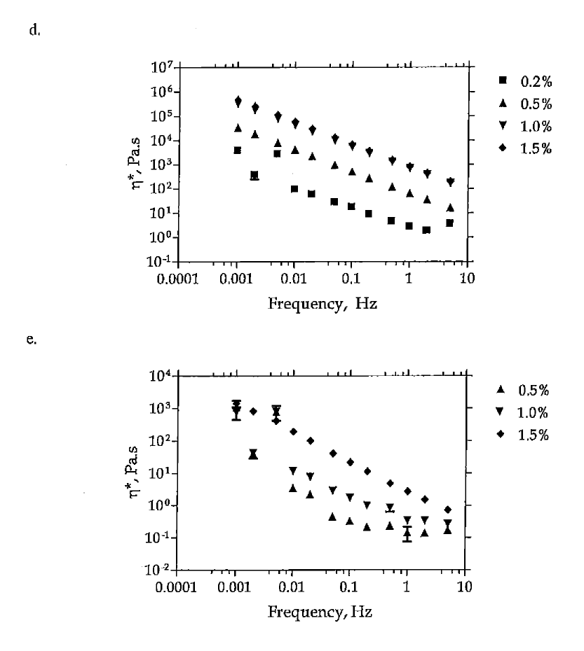

FIGS. 5a to 5e depict the Cox-Merz plots of studied materials. For Avicel RC samples of 0.5 and 1.0% solids, FIG. 4e, as well as 0.2% Cladophora cellulose sample containing 0.1% CMC, FIG. 4d, the log-log relationship between complex dynamic viscosity η* and frequency is non-linear. As previously mentioned, these samples do not exhibit rheological behavior typical for true gel structures.

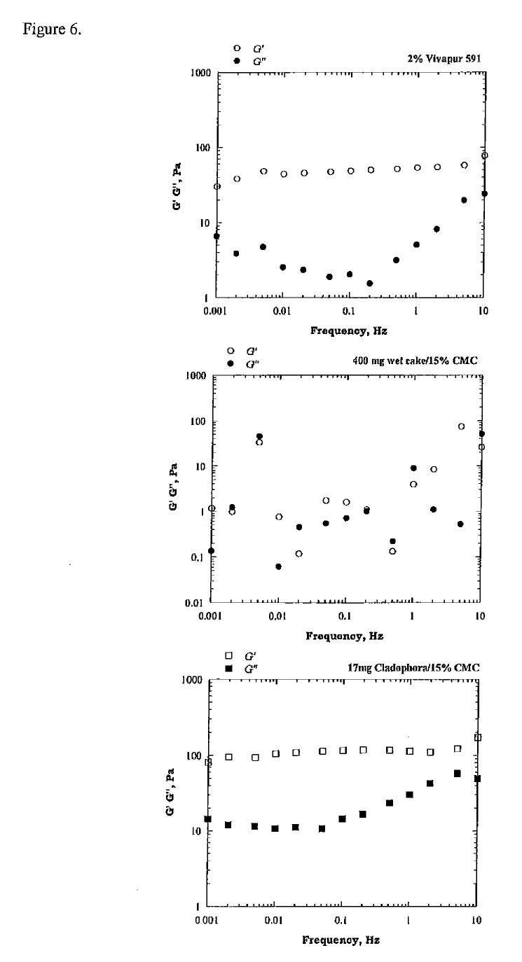

From FIG. 6 it is seen that the properties Cladophora cellulose/CMC gel are compared to Vivapur 591 MCG powder (activated cellulose) and Vivapur MCG wet cake/CMC (non-activated cellulose). The dry solids of content of the Vivapur wet cake and Vivapur 591 corresponded to 2% w/w. It is seen from the plot that Vivapur wet cake, when dispersed with ultrasonic treatment, did not form any gel structures, contrary to Vivapur 591 and Cladophora/CMC samples. Again, a roughly 10 times less concentration of Cladophora/CMC sample is necessary to achieve similar gel strength as that for Vivapur 591.

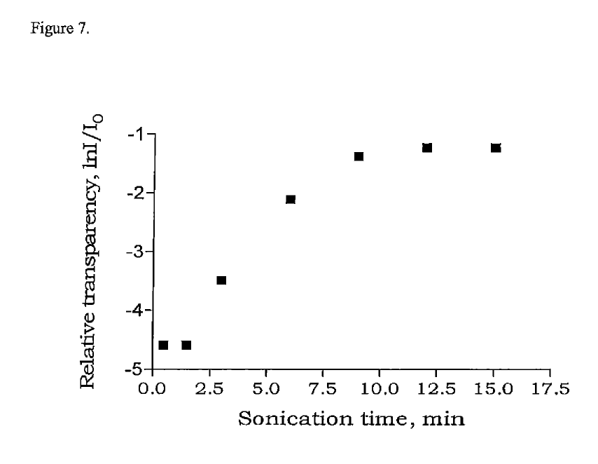

As expected, prolonged ultrasonic treatment resulted in formation of fully activated homogeneous dispersions of cellulose crystallites: In FIG. 7, the relative transparency of Cladophora suspensions increases with the sonication time. Transparency of the resultant dispersion is a beneficial property as it allows higher flexibility with respect to the choice of colorants in the final product.

Cladophora/CMC cellulose dispersion (e.g. 0.5% solids content per volume) does not coagulate even when the sodium chloride content exceeds 10% and up to 50% (weight salt per volume dispersion). The commercial analogues, e.g. Vivapur MCG, JRS Pharma, Germany, coagulate when the sodium chloride content is at 4% (weight salt per volume dispersion) with characteristic phase separation. Even if salt does not totally dissolve, the salt grains remain suspended in the viscous mass, which does not change its appearance.

Cladophora cellulose forms gel structures at cellulose concentrations as low as 0.2% w/v (for all CMC concentrations), whereas the lower threshold for the commercially available analogue is around 1.5% w/v solids contents. Whereas conventional dispersible cellulose grades have commonly been used to reduce oleaginous components in various formulations, e.g. creams or low fat food, their properties have been proved oftentimes unsatisfactory. This is usually the case when substantially fat-free products are desirable: as the fat content is reduced, more cellulose-based ingredients must be added, imparting adverse organoleptic properties. Depending on the product, these adverse effects can include drying sensation, chalkiness, astringent or other disagreeable flavor. It infers from above that fairly high amounts of cellulose-based ingredients are necessary in prior art to achieve marginal fat-like functionality. It has been found in the present invention that by using cellulose of non-seed origin (e.g. algal) it is possible to significantly reduce the concentration of cellulose necessary for formation of stable gel structures and, thereby, reduce negative effects associated with using high amounts of cellulose.

Accordingly, in one embodiment, a gel comprising a non-seed cellulose powder composition may comprise a non-seed cellulose to stabilizing agent weight ratio from about 2:1 to about 40:1. The optimal gel performance is found when the ratio between CMC and MCC is around 1:9, whereas without CMC MCC does not form stable gel structures. In another embodiment, a gel comprising a non-seed cellulose powder composition may comprise a non-seed cellulose to stabilizing agent weight ratio from about 0.2% to about 30% w/v of non-seed cellulose. In yet another embodiment, a gel comprising a non-seed cellulose powder composition may comprise from about 0.5% to about 2% w/v of non-seed cellulose. In yet a further embodiment, a gel comprising a non-seed cellulose powder composition may comprise less than about 0.1% w/v of a stabilizing agent.

The cellulose in the present invention has a non-seed organism origin. It is characterized by large surface area typically >5 m²/g as obtained by BET N₂ gas adsorption analysis and pore volume >0.01 cm³/g. It is a stable, highly crystalline powder capable of retaining its highly porous structure of its particles even in highly moist environments (RH ˜100%) or during drying, e.g. spray-drying. When dispersed alone or in combination with stabilizing agents such as hydrocolloids (e.g. CMC) in water, the material in the present invention produces stabile gel structures. The lower threshold for exhibiting gel-like properties is around 0.2% w/v.

The potential fields of application include frozen dairy comestibles (e.g. ice-cream, ice-milk, yoghurt, mayonnaise, etc), topically applied compositions, various pharmaceutical dispersive systems (e.g. creams, ointments, suspensions, emulsions) as well as topical preparations for cosmetic use. In addition, algal and bacterial cellulose exhibit many unique properties including high mechanical strength, high crystallinity, and ultra-fine nanofibril network structure of high porosity useful in designing biocompatible artificial tissue structures, e.g. artificial blood vessel, skin and bone structures. Bacterial cellulose from Acetobacter xylinum has previously been disclosed as a potential substrate for such biological tissue engineering (see G. Helenius, H. Bäckdahl, A. Bodin, U. Nannmark, P. Gatenholm, B. Risberg. 2006. “In vivo biocompatibility of bacterial cellulose”, Journal of Biomedical Materials Research Part A, 76A (2): 431-438; A. Bodin, L. Gustafsson, P. Gatenholm 2006. “Surface-engineered bacterial cellulose as template for crystallization of calcium phosphate.” Journal of Biomaterials Science Polymer Edition, 17(4):435-477; H. Bäckdahl, G. Helenius, A. Bodin, U. Nannmark, B. R. Johansson, B. Risberg, P. Gatenholm. 2006. “Mechanical properties of bacterial cellulose and interactions with smooth muscle cells”, Biomaterials, 27: 2141-2149). Accordingly, cellulose of non-seed origin can also be used as a suspending aid in production of various types of paints and dyes. Further, non-seed cellulose compositions may be used in a biocompatible material for artificial tissue engineering or in an implantable biomaterial.

### EXAMPLES

### Example 1

### Cream Formulation Containing Hydrocortisone Acetate

| | %, w/w |
|---|---|
| Aqueous phase | |
| Cladophora/CMC dispersion* | To 100% |
| Methylparaben | 0.25 |
| Hydrocortisone acetate | 1 |
| Propylene glycol | 10 |
| Polysorbate 80 | 5 |
| Oleaginous phase | |
| Cetyl alcohol | 2.5 |
| Propylparaben | 0.15 |
| Glyceryl monostearate | 10.0 |

*Cladophora/CMC, Blanose 7MF (85/15% w/w cellulose/CMC ratio) dispersion containing e.g. 0.5 to 1% w/w Cladophora.

The oleaginous phase components are mixed separately and heated to 70° C. The aqueous phase components are dispersed in water using a high-shear homogenizer until the Cladophora cellulose is fully activated. The hot oleaginous phase is then poured into aqueous phase and thoroughly mixed. The hot creams are poured into ointment tubes and allowed to solidify.

### Example 2

### Thermostable Fat-Free Flavored Cookie Filling

| Ingredient | %, w/w |
|---|---|
| Cladophora/CMC dispersion* | To 100% |
| Glycerin | 20 |
| Sugar, Powdered | 40 |
| Natural flavor | Variable |
| Colorants | Variable |

*Cladophora/CMC, Blanose 7MF (85/15% w/w cellulose/CMC ratio) dispersion containing e.g. 0.5 to 1% w/w Cladophora.

Disperse Cladophora/CMC, sugar, colorants, and flavors in water until cellulose is fully activated. Heat glycerin to 60° C. and added to the dispersion under stirring. Mix thoroughly into to a homogeneous jelly like mass.

### Example 3

### Biocompatible Cellulose-Based Substrate For Artificial Blood Vessel Engineering

Sterilize Cladophora by repeated boiling in Millipore™ water and subsequent autoclaving for about 30 minutes. Activate the resultant Cladophora cellulose nanofibrils aseptically in Millipore™ water to produce a thick gel structure and dry the latter on a cylindrical mould to produce a cellulose tube. Repeat the procedure manifold so as to produce tubes of desired thickness.

### Example 4

### Biocompatible Cellulose Based Substrate For Artificial Bone Engineering

Sterilize Cladophora by repeated boiling in Millipore™ water and subsequent autoclaving for about 30 minutes. Activate aseptically the resultant Cladophora cellulose nanofibrils in Millipore™ water to form a thick gel structure. Add sterilized calcium phosphate to dispersion and rigorously stir. Dry the resultant mass to moisture content of about 5 wt %. Mould the mass into desired shape via direct compression.

| Ingredient | %, w/w |
|---|---|
| Cladophora cellulose powder | 4 |
| Calcium phosphate | 20 |
| Millipore TM Water | To 100% |

1-13. (canceled)

14. A gel comprising a non-seed cellulose powder composition, wherein the non-seed cellulose is made from algae, fungi or tunicates.

15. A suspension comprising a non-seed cellulose powder composition, wherein the non-seed cellulose is made from algae, fungi or tunicates.

16. A gel comprising a non-seed cellulose powder composition and a stabilizing agent, wherein the non-seed cellulose is made from algae, fungi or tunicates, and wherein the non-seed cellulose powder composition comprises a non-seed cellulose to stabilizing agent weight ratio from about 2:1 to about 40:1.

17. The gel according to claim 14, comprising from about 0.2% to about 30% w/v of non-seed cellulose.

18. The gel according to claim 17, comprising from about 0.5% to about 2% w/v of non-seed cellulose.

19. The gel according to claim 14, comprising less than about 0.1% w/v of a stabilizing agent.

20. A food product comprising the gel of claim 14.

21. A topically applied composition comprising the gel of claim 14.

22. A pharmaceutical formula comprising the suspension of claim 15.

23. A paint formula comprising the suspension of claim 15.

24. (canceled)

25. An implantable biomaterial comprising the gel of claim 14.

26. (canceled)

27. (canceled)

28. (canceled)

29. (canceled)

30. (canceled)

31. (canceled)

32. A food product comprising the suspension of claim 15.

33. A pharmaceutical formula comprising the gel of claim 14.

34. The gel of claim 14, wherein the non-seed cellulose is made from algae.

35. The gel of claim 34, wherein the algae comprises green algae from Cladophorales order, Siphonocladales order, or combinations thereof.

36. The gel of claim 34, further comprising a stabilizing agent comprising a hydrocolloid.

37. The suspension of claim 15, further comprising a stabilizing agent comprising a hydrocolloid.

38. A method of forming the gel of claim 14, comprising dispersing the non-seed cellulose powder composition in aqueous medium with sufficient energy to form a gel.

39. The method of claim 38, wherein said dispersing comprises using a high-shear homogenizer.

40. The method of claim 38, wherein said non-seed cellulose powder composition is present in an amount of from about 0.2% to about 30% w/v of non-seed cellulose.
